(19) United States
(12) Patent Application Publication
(10) Pub. No.: US 2022/0040027 A1
(43) Pub. Date: Feb. 10, 2022

(54) CONTROL AND SENSOR SYSTEM FOR DEVICES ASSISTING IN JOINT FLEXION

(71) Applicant: XZO, Inc., Boulder, CO (US)

(72) Inventors: Jose Francisco Catarino Palomo, Denver, CO (US); Jose Miguel Rosales Yanez, Aurora, CO (US)

(73) Assignee: XZO, Inc., Boulder, CO (US)

(21) Appl. No.: 17/397,645

(22) Filed: Aug. 9, 2021

Related U.S. Application Data
(60) Provisional application No. 63/063,212, filed on Aug. 7, 2020.

(51) Int. Cl. A61H 1/02; G16H 20/30; G16H 40/63

(52) U.S. Cl. CPC A61H 1/0288; G16H 20/30; G16H 40/63; A61H 2201/5007; A61H 2201/165; A61H 2201/5061; A61H 2201/1638

(57) ABSTRACT

Embodiments of the present disclosure are directed to devices for assisting joint flexion. Devices for assisting joint flexion can include a wearable component configured to fit over a user's finger, an actuator interface positioned at a joint of the user's finger, and a force sensor on a palmar side of a user's finger joint on an exterior surface of the wearable component. A force sensor measures an overall force applied to an object by a contact surface of the wearable component. Such devices can also include a motor coupled with the wearable component that provides the mechanical driving force to the actuator interface to assist with flexion of the user's finger joint.

### CROSS REFERENCE TO RELATED APPLICATIONS

This application claims the priority benefit of U.S. Provisional Application No. 63/063,212 filed Aug. 7, 2020, pending, which is incorporated herein by reference in its entirety.

### TECHNICAL FIELD

Embodiments relate generally to devices for assisting users with joint flexion.

### BACKGROUND

Many individuals have limited strength and mobility in their joints. Various medical ailments can lead to a patient's reduced capacity to flex and extend their joints without external assistance, such as temporary injuries, illnesses, chronic diseases, and congenital disabilities. Examples of conditions that impact joint mobility and strength include stroke, brain tumors, peripheral neuropathy, arthritis, and cerebral palsy.

Some individuals can normally flex and extend their joints but may desire to amplify their joint strength and gripping capacity. Joint and grip strength are implicated in athletic settings such as weightlifting, resistance training, gymnastics, baseball, and golf. Additionally, wear and tear is applied to joints in occupational settings involving the repetitive lifting of heavy objects such as boxes, luggage, and construction materials.

### SUMMARY

Embodiments of the present disclosure are directed to devices for assisting joint flexion. Devices for assisting joint flexion can include a wearable component configured to fit over a user's finger, an actuator interface positioned at a joint of the user's finger, and a force sensor on a palmar side of a user's finger joint on an exterior surface of the wearable component. A force sensor measures an overall force applied to an object by a contact surface of the wearable component. Such devices can also include a motor coupled with the wearable component that provides the mechanical driving force to the actuator interface to assist with flexion of the user's finger joint. In one embodiment, the control system of the device is connected to the force sensor and the motor. The control system is configured to receive a first signal from the force sensor indicating an overall force applied to the object by the contact surface and to receive a second signal indicating a current applied to the motor. The device-applied force is determined by the control system, based on the current applied to the motor, according to some embodiments. The user-applied force is determined by the control system, based on the overall force and device-applied force, according to some embodiments. The control system continues to drive the motor to flex the actuator interface until the user-applied force to the object decreases, according to some embodiments.

### BRIEF DESCRIPTION OF THE DRAWINGS

FIG. 1 illustrates a block diagram of the individual components in the device for assisting joint flexion, according to embodiments of the present disclosure.

FIG. 2A illustrates a palm side view of a device for assisting joint flexion worn on a user's hand, according to embodiments of the present disclosure.

FIG. 2B illustrates a back side view of a device for assisting joint flexion worn on a user's hand, according to embodiments of the present disclosure.

FIG. 3A illustrates a palm side view of a device for assisting joint flexion worn on a user's hand, according to embodiments of the present disclosure.

FIG. 3B illustrates a back side view of a device for assisting joint flexion worn on user's hand, according to embodiments of the present disclosure.

FIG. 4A illustrates a palm side view of a device for assisting with joint flexion of the user's individual fingers using metal band actuator interfaces, according to embodiments of the present disclosure.

FIG. 4B illustrates a dorsal view of a device for assisting with joint flexion of the user's individual fingers using metal band actuator interfaces, according to embodiments of the present disclosure.

FIG. 5 illustrates a side view of a metal band actuator interface for a device assisting with joint flexion of the user's individual fingers, according to embodiments of the present disclosure.

FIG. 6 illustrates a process flow diagram for calibration and use of a device for assisting joint flexion in the user's hand, according to embodiments of the present disclosure.

FIG. 7 illustrates a control system of a device for assisting joint flexion in the user's individual fingers, according to embodiments of the present disclosure.

FIG. 8 illustrates a process flow diagram describing calibration and use of the device for assisting joint flexion in the user's hand, according to embodiments of the present disclosure.

FIG. 9 illustrates an embodiment of the device for assisting joint flexion worn on user's hand with an external pack, according to embodiments of the present disclosure.

Corresponding reference characters indicate corresponding parts throughout the several views.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Embodiments of the present disclosure include devices for assisting joint flexion. Some device embodiments utilize strain gauge based-sensors and a control system to detect the force a user is applying to an object. Joint flexion devices using strain gauge based-sensors may be more affordable to consumers than traditional external assistive devices utilizing myoelectric sensors. In some embodiments, one or more of the force sensors spans the interphalangeal and/or metacarpophalangeal joint for a particular digit. Embodiments of joint flexion devices can include devices for assisting joint flexion in hinge joints, including fingers, hands, elbows, knees, toes, ankles, as well as devices assisting with two-dimensional range of motion in ball-and-socket joints, or other joints of the body.

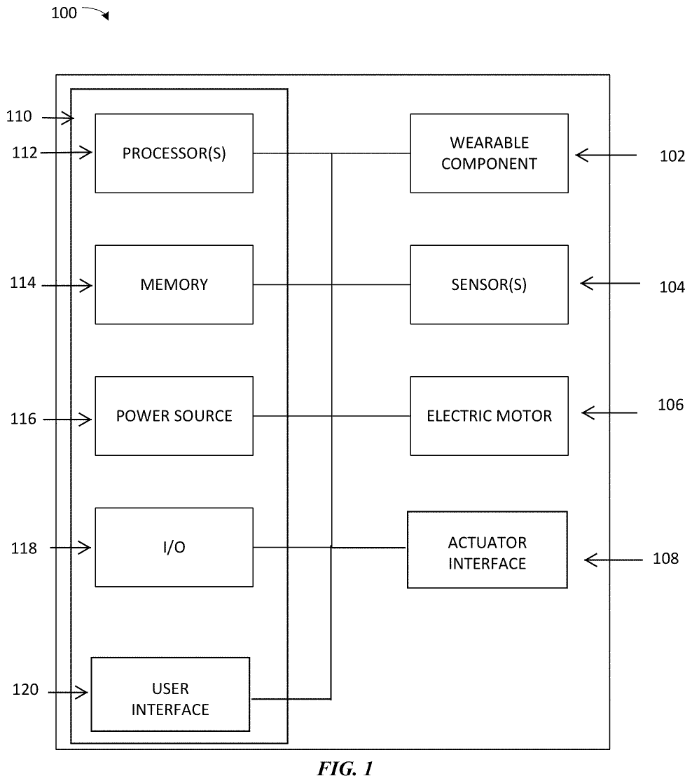

As shown in FIG. 1, a device 100 for assisting with joint flexion includes a wearable component 102, a sensor 104, a motor 106, an actuator interface 108, and a control system 110, 700. The control system 110, 700 includes a processor 112, memory 114, a power source 116, an input/output (I/O) 118, and a user interface 120. As shown in FIG. 1, each of the components 102, 104, 106, 108, 110, 112, 114, 116, 118, and/or 120 may be communicably and/or electronically coupled to one another, for example by one or more wires or buses.

According to some embodiments, the hardware components 104-120 of the device 100 are connected to the user with a wearable component 102. The wearable component 102 can include straps or rings made of Velcro, 3-D printed polymers, silicone, rubber or metal. Exterior gloves, sleeves, and braces embedded with the hardware components 104-120 can also serve as the wearable component 102.

Embodiments of the device 100 include at least one sensor 104 located on the flexion side of a user's joint. The sensor 104 can include strain gauges that are used to detect an overall force signal applied both by the device 100 and the user to an object. An individual sensor 100 can be connected to the control system 110 though circuitry such as a Wheatstone bridge circuit coupled to an amplifier, which amplifies the detected overall force signal to be read in by the processor 112.

According to some embodiments, the device 100 contains a motor 106 connected to the control system 110 and to at least one actuator interface 108 located on at least one of the user's joints. The motor 106 can be, but is not limited to, a servomotor, stepper motor, DC motor, AC motor, brushless motor, or linear actuator, for example. The actuator interface 108 attached to the motor 106 can be located on either the flexion or extension side of the user's joint. Strings, metal bands attached to lead screws, and gears are a few examples of actuator interfaces 108. Actuator interfaces 108 with metal bands or gears may also include additional small electric motors.

In addition to the Wheatstone bridge circuit and amplifier, the control system 110 can include a sensing integrated circuit and processor 112. The sensing integrated circuit measures the current from the motor 106. The current value is sent to the processor 112 and utilized to determine the device-applied force. The processor 112 performs a curve-fitting algorithm to determine the device-applied force. The processor 112 can perform curve-fitting algorithms such as linear, quadratic, logarithmic, and exponential algorithms. In the preferred embodiment, the processor 112 performs an exponential least squares curve fitting algorithm to determine the device-applied force. The processor 112 may perform a machine learning algorithm to determine the forces applied the device and the user.

The control system 110 for the device 100 includes memory 114, a power source 116, input/output interface 118, and a user interface 120. Memory 114 for the device 100 can be random access memory. Rechargeable power sources as well as power sources that can be plugged into an outlet are some examples of power sources 116 that can be used. Input/output interface 118 for the device may wirelessly communicate with an application on a user's smartphone or recognize user voice data to implement voice commands for the device 100. Displays, buttons, and touch screens are examples of potential user interfaces for the device 120. Input/output settings the user may adjust using the input/output interface 118 include device functional mode, maximum grip strength provided by the device, opening and closing speed of the device, and force sensor sensitivity.

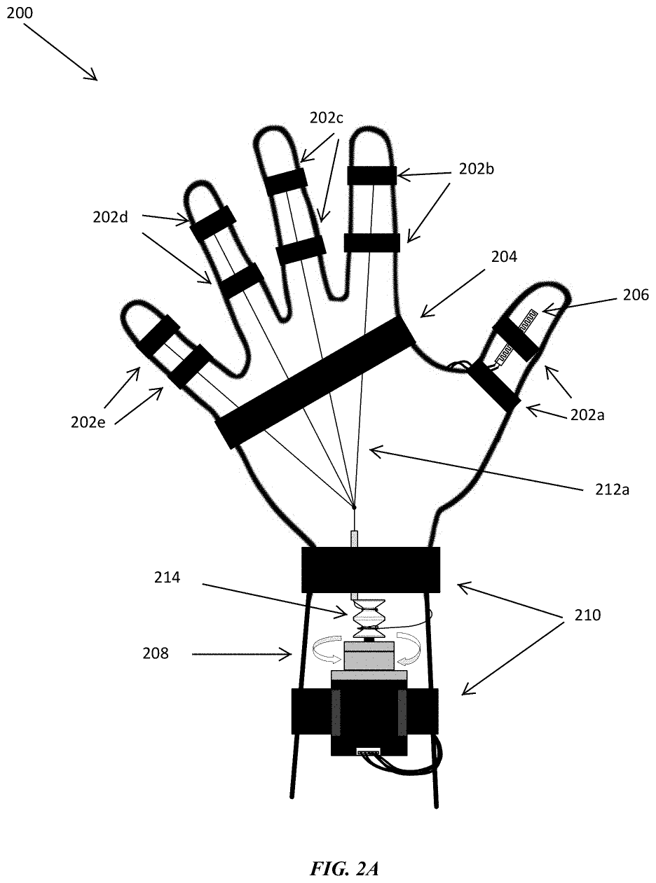

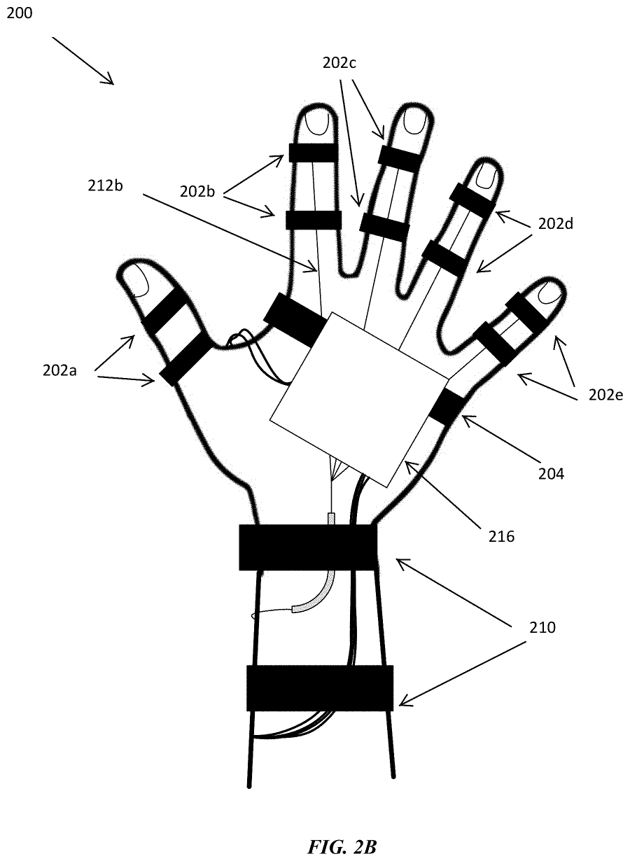

FIG. 2A shows a device 200 that assists joint flexion in the hand by applying a force to one or more of the user's fingers to help the user open and close their fingers. Fingers in this disclosure is used to refer to the user's fingers as well as the user's thumbs. The wearable components 202 can be straps or attachments configured to couple with the user's hand, wrist, and/or forearm. Wearable components 202a-e can be strap segments of the wearable component located on each individual finger. In some cases, the wearable component 202 can include a strap that is configured to be worn around a portion of the finger adjacent to a joint of the finger. In other cases, the wearable components 202 can include multiple straps that are worn around different portions of a finger. The guide band 204 can be located around the user's palm and assists with opening and closing the user's hand. For example, the guide band 204 can route or guide an actuator interface 212a along the surface of the hand such that when the motor is activated to pull or push the actuator interface 212a, the guide band 204 can cause the actuator interface 212a move the wearable component 202 in a defined motion that aids opening or closing of the finger joint. The strain gauge-based force sensor 206 can be located on the palmar side of the user's thumb interphalangeal joint. The force sensor 206 can be connected to the control system through a circuit such as a Wheatstone bridge circuit and an amplifier. The control system and the motor 208 are located on the dorsal side of the user's hand. The motor 208 can be attached to the user with wearable components 210a-b located on the user's wrist and forearm and is a stepper motor. The actuator interface 212a can be a system of strings on the palmar side of the user's hand connected to the wearable components 202, guide band 204, and motor 208. The strings of the actuator interface 212a are wound around the spool 214 or the motor 208. The user's hand can be opened or closed depending on the direction the motor 208 spins the spool 214.

FIG. 2B illustrates a back side view of the device 200 worn on a hand of a user. The control system 216 can be attached to the guide band 204 and rest on the top of the user's hand. Control system 216 may be or include control system 110 as described above. The actuator interface 212b can include a system of strings on the back side of the user's hand connected to the wearable components 202, guide band 204, and motor 208. The motor 208 can aid opening of the hand by tensioning the actuator interface 212b to help open one or more finger joints. When the motor 208 is being driven to help close the hand, the motor 208 can extend or release tension in the actuator interface 212b thereby allowing the user's fingers to close.

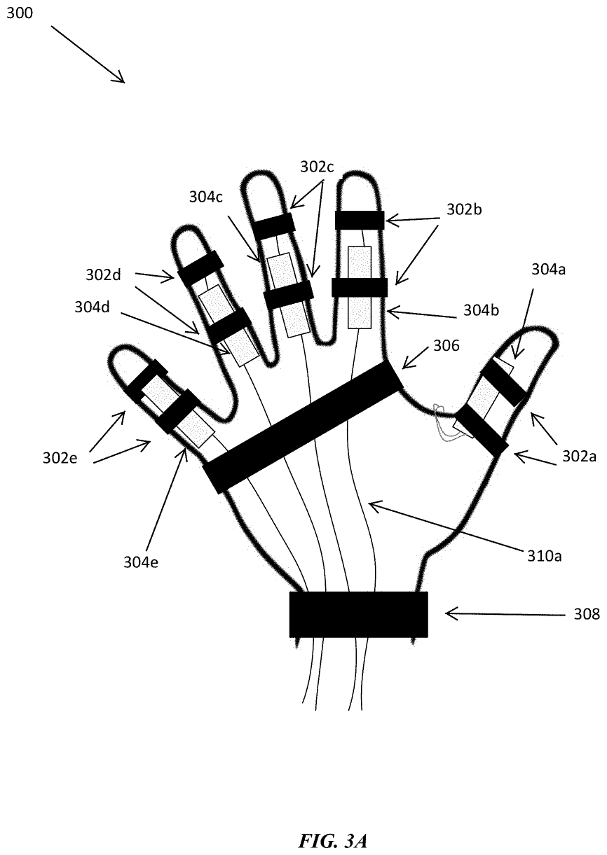

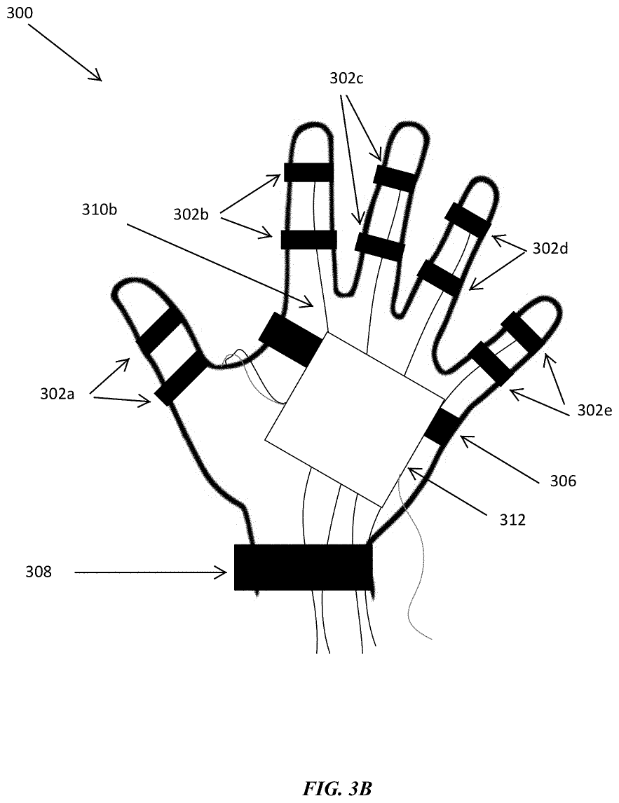

FIG. 3A shows the palm side view of a device 300 for assisting with joint flexion of the user's individual fingers. The device 300 can include wearable components 302a-e that are straps located at the user's interphalangeal finger joints. In some embodiments strain sensors 304a-e can be located on each of the user's five fingers at the user's interphalangeal and/or metacarpophalangeal joints. The strain-gauge sensors 304 can be configured to measure a force applied by each finger to an object. In some cases, the force measured by the strain sensors 304 includes both the force the user is applying to the object as well as the force the device 300 is applying to the object. By having strain gauge sensors 304 on each finger, the device 300 can individually control each finger based on the force applied by each finger. The guide band 306 can be located around the user's palm and assists with the flexion and extension of the user's fingers. Wearable component 308 can include a strap around the user's wrist to secure the device 300 to the user. The actuator interface 310a can include a system of strings on the palm side of the user's hand and be connected to the wearable components 302, guide band 306, and electric motor 208. The actuator interface 310a can be coupled to a motor such that actuation of the motor caused the actuator interface to shorten thereby helping close the fingers or lengthen thereby allowing the fingers to open.

FIG. 3B shows the back-side view of the device 300 for assisting with joint flexion of the user's individual fingers. The control system 312 can be attached to the guide band 306 and rests on the top of the user's hand. The actuator interface 310b can be a system of strings on the dorsal side of the user's hand connected to the wearable components 302, guide band 306, and electric motor 204. The force measurements detected by the multiple strain gauge sensors 304 (shown in 3A) determine the force output of the device 300 and motor 204. The actuator interface 310b implements the force output of the device 300 and motor 204 and can open and close individual fingers based on the force measurements detected by the multiple strain gauge sensors 304 (shown in 3A).

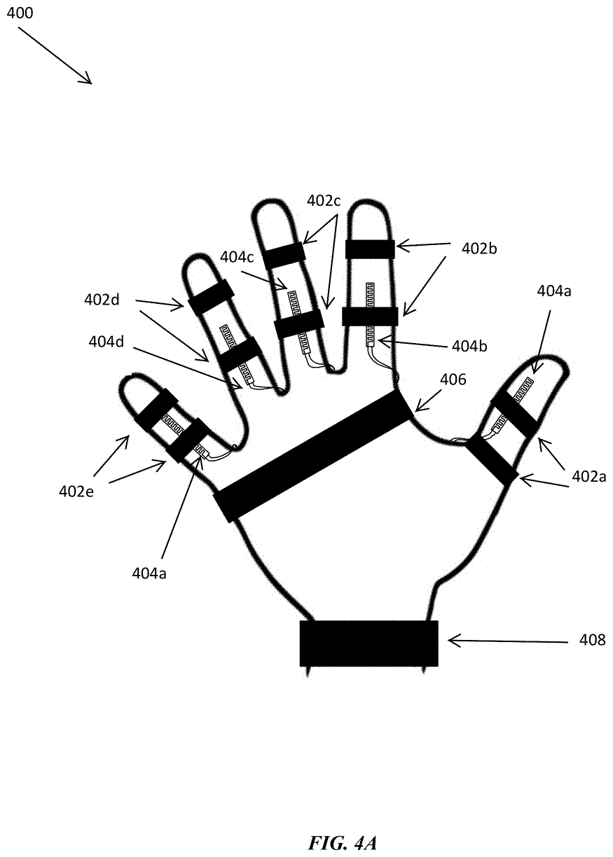

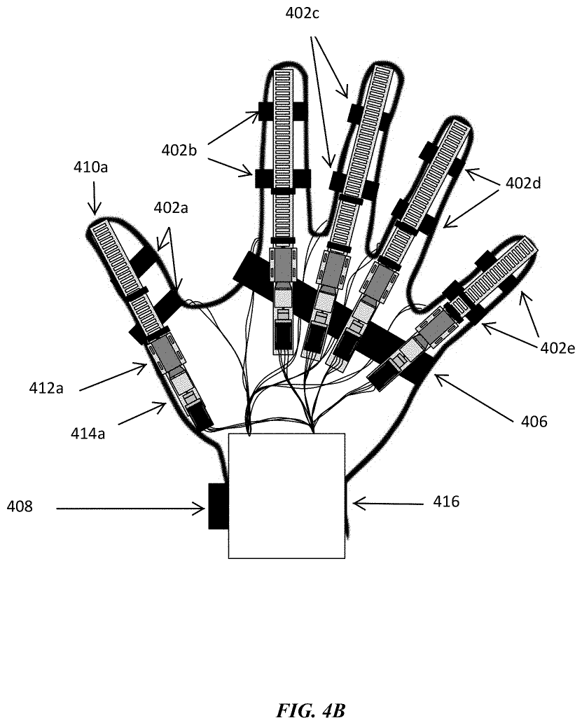

FIG. 4A shows the palm side view of the device 400 for assisting with joint flexion of the user's individual fingers, using metal band actuator interfaces. The wearable components 402a-e can be straps that are located at the user's interphalangeal and/or metacarpophalangeal finger joints. Strain-gauge sensors 404a-e can be located on each of the user's five fingers at the user's interphalangeal joints. The sensors 404 can be configured to measure the force applied by each finger. The force measurements from each strain-gauge sensor 404a-e can be used to control an individual finger. The multiple force measurements from the set of strain-gauge sensors 404a-e can be used to derive an overall force applied by the hand. The guide band 406 can be located around the user's palm and assists with the flexion and extension of the user's fingers. Wearable component 408 is a strap around the user's wrist to secure the device 400 to the user, for example.

FIG. 4B shows back side view of the device 400 for assisting with joint flexion of the user's individual fingers. Device 400 may be worn on a user's hand. The device 400 can include metal band actuator interfaces 410. The metal band actuator interface 410 can be a set of metal bands coupled to the back side of the user's hand and connected to the wearable components 402, guide band 406, finger band drivers 412, small-geared stepper motors 414, and motor 204. The control system 416 is attached to the guide band 406 and rests on the top of the user's hand. For example, the small-geared stepper motor 414 includes a finger band driver (drive gear) 412 that interfaces with the metal band actuator interface 410. When the small-geared stepper motor 414 rotates the finger band driver (drive gear) 412 in a first rotational direction, the drive gear 412 causes the metal band actuator interface 410 to move in a first linear direction. When the small-geared stepper motor 414 rotates the finger band driver (drive gear) 412 in a second rotational direction, the drive gear 412 causes the metal band actuator interface 410 to move in a second linear direction. The metal band actuator 410 interfaces can be coupled adjacent to joints on the finger such that the linear motion of the metal band actuator interface 410 can help open or close the finger. Each finger can be controlled independently.

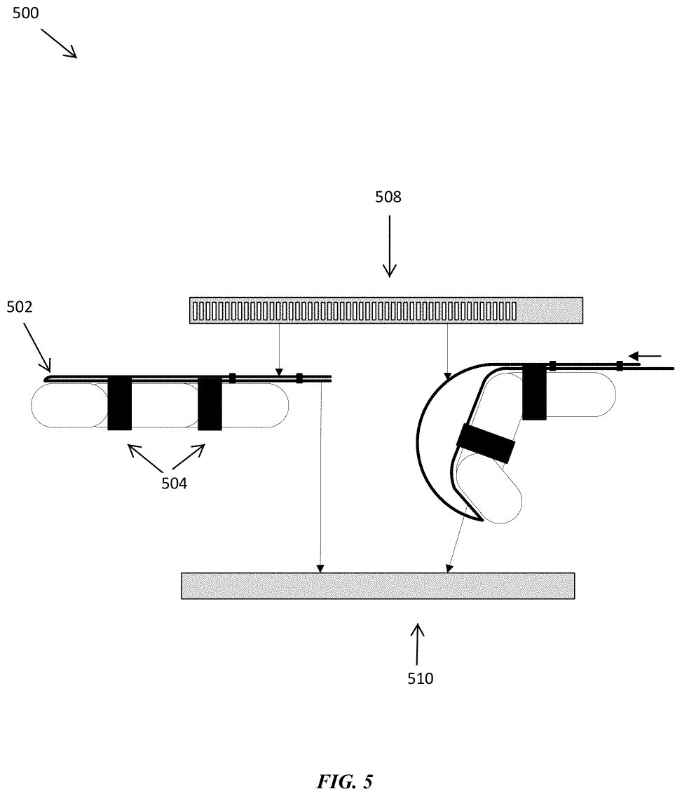

FIG. 5 shows a side view of the metal band actuator interface 502 for the device 500 assisting with joint flexion of the user's individual fingers. The metal band actuator interface 502 is attached to the user's fingers by the wearable components 504. The top 508 of the metal band actuator interface 502 is threaded and the bottom 510 of the metal band actuator interface is smooth. The metal band actuator interface 502 is looped on top of the user's finger. When the metal band actuator interface 502 is driven by the finger band drivers 412 in a first direction it causes the user's finger to curl or rotate closed. When the metal band actuator interface 502 is driven by the finger band drivers 412 in a second direction it causes the user's finger to open or straighten.

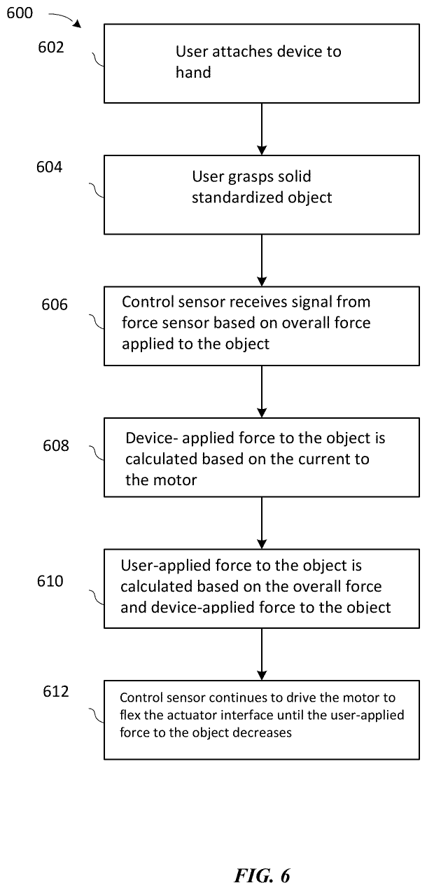

FIG. 6 illustrates a process flow 600 describing calibration and use of the devices described herein (e.g., devices 100, 200, 300, 400, 500, 900) for assisting joint flexion in the user's hand. At 602, a user can attach the device to their body using a wearable component. At 604, the user can grasp a solid object. At 606, the control system of the device receives a signal from the force sensor based on the overall force applied to an object. At 608, the device-applied force to the object can be determined based on current applied to the motor. At 610, the user-applied force to the object can be determined by the control system based on the overall force and the device-applied force. At 612, the control system can continue to drive the motor to flex the actuator interface until the user-applied force to the object decreases.

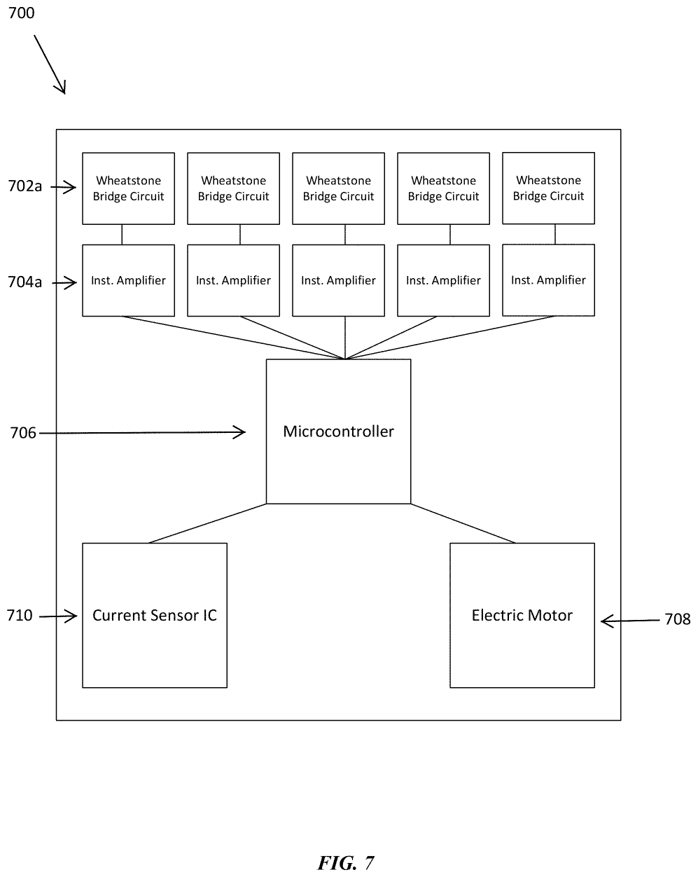

FIG. 7 is a diagram of the control system 700 for the devices described herein (e.g., devices 100, 200, 300, 400, 500, 900) for assisting with joint flexion of the user's individual fingers as described herein. The force sensors on the individual fingers can each be attached to a Wheatstone bridge circuit 702a-e. The Wheatstone bridge circuits 702a-e can be coupled to a set of instrumental amplifiers 704a-e. The set of instrumental amplifiers 704a-e connect to the microcontroller 706. The microcontroller 706 can use the overall force input from the force sensors 304a-e to generate the necessary output from the motor (mechanism driver) 708 for the device to assist with joint flexion. For example, the microcontroller 506 can use the overall force input from the force sensors 304a-e to generate the necessary output from the motor (mechanism driver) 708 to drive the actuator interfaces to open or close the user's fingers. The sensing integrated circuit 710 measures the current of the motor 708 and sends a current measurement signal to the microcontroller 706. The microcontroller 706 uses the current measurement from the motor 708 to calculate the force applied by the device 300.

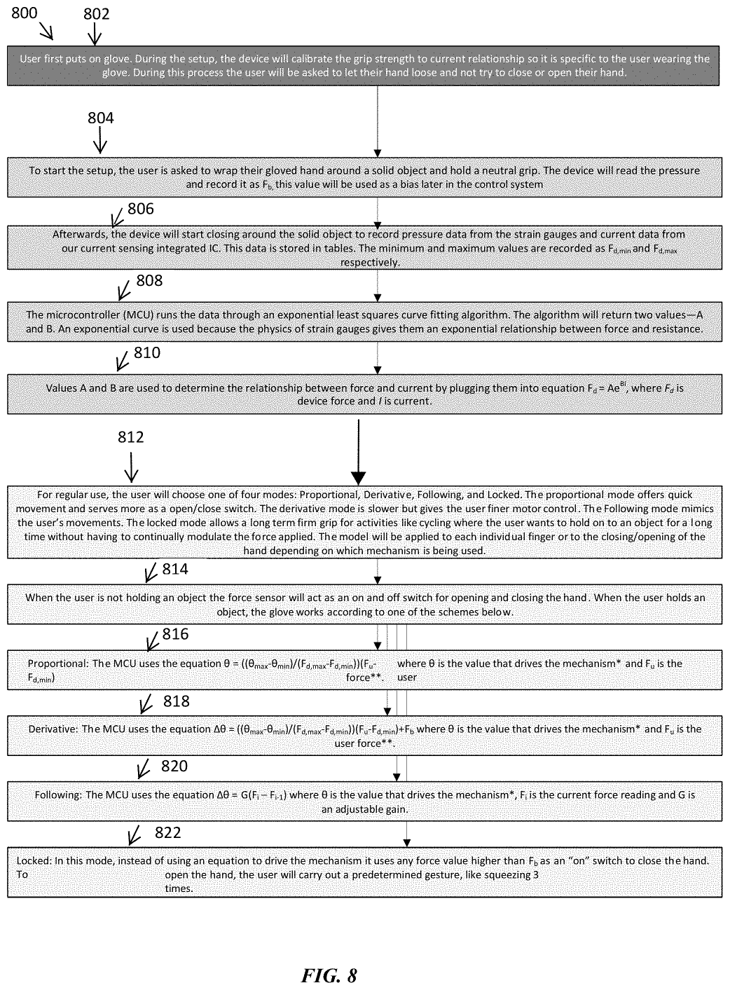

FIG. 8 illustrates a process flow 800 describing the calibration and use of the devices described herein (e.g., devices 100, 200, 300, 400, 900) for assisting joint flexion such as the device described herein. At 802, to calibrate the device for an individual user, the user can attach the device to their hand. At 804, the user wearing the device can grasp a solid standardized object and the device will record the base overall force measurement as F_{b}. At 806, the device can close around the object and the force data from the sensors and current data detected by the sensing integrated circuit can be stored by the microprocessor in tables. At 808, the microcontroller can run the force and current data through an exponential least squares curve fitting algorithm to determine important scalar values (A and B) that will be applied to determine the force applied solely by the device (F_{d}). At 810, the device force is calculated from the equation F_{d}=Ae^{BI}, where I is the current.

At 812, a user can choose between the device's functional modes. According to some embodiments, the device includes one or more of four functional modes: Proportional Mode, Derivative Mode, Following Mode, and Locked Mode. When the device is in Proportional Mode, the device closes the hand an amount proportional to the amount of user-applied pressure. The Proportional Mode allows for the device to move the user's hand quickly. In Derivative Mode, the device closes the hand based on the change in user pressure from the base overall force measurement (F_{b}). Derivative Mode allows for finer motor control by the user. Following Mode has the most sensitivity to the user's movements, as the device moves according to the change in user force from the last user force reading. At 814, the hand will begin opening once user-applied force decreases below the base force value (F_{b}) in Proportional Mode, Derivative Mode, and Following Mode. Locked Mode allows for long-term closed grip, where the user does not have to continuously apply force, according to some embodiments. At 822, a user can perform a predetermined gesture such as, for example, squeezing the hand three times for the device to open the hand.

At 816, the microprocessor can determine a current value for driving the motor in the Proportional mode using the following equation: θ=((θₘₐₓ−θₘᵢₙ)/(F_{d max}−F_{d min})) (Fᵤ−F_{d min}) where θ is the value that drives the mechanism and Fᵤ is the user force. The user force is calculated by Fᵤ=F−F_{d}, where F is the overall force of the system and Fd is the device force.

At 818, the microprocessor can determine a current value for driving the motor in the Derivative Mode using the following equation: Δθ=((θₘₐₓ−θₘᵢₙ)/(F_{d max}−F_{d min})) (Fᵤ−F_{d min})+F_{b}, where θ is the value that drives the mechanism and Fᵤ is the user force.

At 820, the microprocessor can determine a current value for driving the motor in the Following Mode using the following equation: Δθ=G(Fᵢ−Fᵢ₋₁), where θ is the value that drives the mechanism, Fᵢ is the current force reading and G is an adjustable gain.

A grip strength training mode may be included in some embodiments. In grip strength training mode, the user will select a resistance setting displayed in pounds, and the selection will adjust the force applied by the device, according to some embodiments. The user will apply pressure and try to exceed the set force applied by the device in order to improve their grip strength ability. If the user pressure exceeds the set force applied by the device, the force applied by the device will increase incrementally until the user cannot apply pressure that exceeds the force applied by the device, for example. This pressure value can be stored as the user's maximum grip strength. One version of the grip training mode can set the force applied by the device at 80% of the user's maximum grip, and the force applied by the device may be gradually increased over a period of time selected by the user, according to some embodiments.

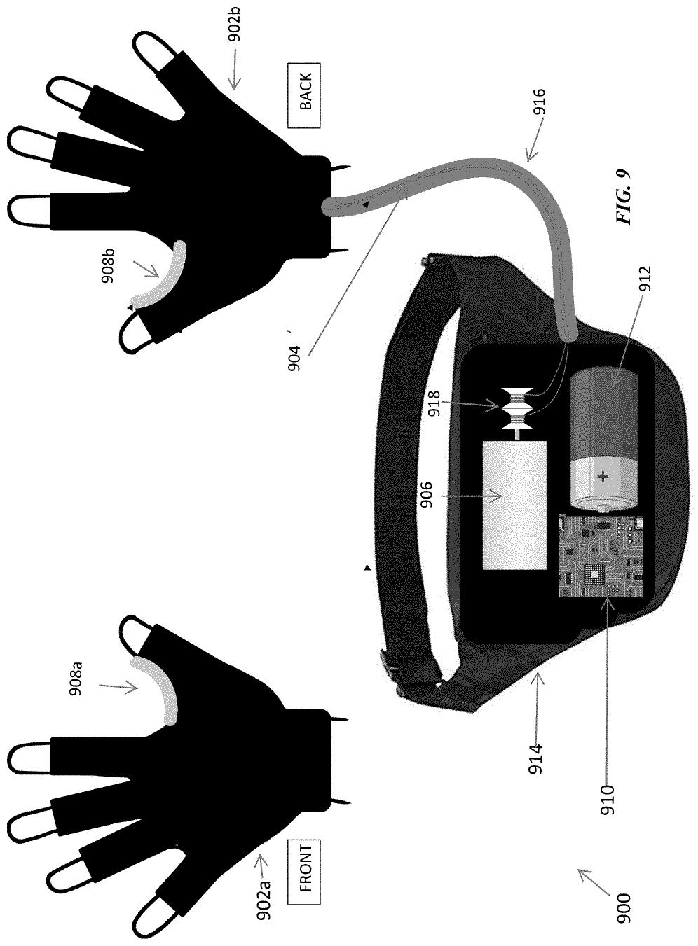

FIG. 9 shows a device 900 that assists joint flexion in the hand by applying a force to one or more of the user's fingers to help the user open and close their fingers, according to embodiments of the present disclosure. The wearable components 902a-b can be gloves with internal finger bands located at the user's interphalangeal finger joints. The internal finger bands of the wearable component are located on each individual finger. In some cases, the internal finger bands can include a single strap that is configured to be worn around a portion of the finger adjacent to a joint of the finger. In other cases, the internal finger bands can include multiple straps that are worn around different portions of a finger. The wearable components 902a-b can include an internal guide band that can be located around the user's palm and assists with opening and closing the user's hand. For example, the internal guide band can route or guide an actuator interface 904 along the surface of the hand such that when the motor 906 is activated to pull or push the actuator interface 904, the guide band can cause the actuator interface 904 move the wearable component 902 in a defined motion that aids opening or closing of the finger joint. The strain gauge-based force sensors 908a-b can be located on the palmar side of the user's thumb interphalangeal joint. The force sensor 908a-b can be connected to the control system 910 through a circuit such as a Wheatstone bridge circuit and an amplifier. In one embodiment, the control system 910, power source 912 and the motor 906 are located in an external pack 914 worn or carried by the user. The external pack can be worn on the user's waist, carried like a bag over their shoulder, strapped on a user's limb, and/or attached to a user's medical equipment such as a wheelchair or walker, according to some embodiments. The device motor 906 can be a stepper motor. The actuator interface 904 can be a system of strings on the palmar side of the user's hand connected to the wearable components 902a-b, guide band, and motor 906. A portion of the actuator interface may be encased in a tube 916 connecting between the wearable component 902a-b and the external pack 914. The tube 916 can have external straps that can attach to the user or the user's medical device in order to minimize catching or snagging of the tube on other objects. A barrel adjuster, or in some case a Bowden cable system, is located on at least one end of the tube 916 in order to maintain tension in the strings of the actuator interface 904. The strings of the actuator interface 904 are wound around the spool 918 or the motor 906. The user's hand can be opened or closed depending on the direction the motor 906 spins the spool 918.

What is claimed is:

1. A device for assisting joint flexion, comprising:
a wearable component configured to be worn on a first finger of a user adjacent to a joint of the first finger;
at least one force sensor located on a palm side of a second finger of the user, the force sensor operative to measure a first force applied to an object by the user;
a motor coupled with the wearable component and operative to apply a second force to the wearable component;
a control system connected to the at least one force sensor and to the motor, the control system configured to:
receive a first signal from the at least one force sensor indicating the first force applied to the object by the user;
receive a second signal indicating a current applied to the motor;
determine the second force applied by the device to the object based on the current applied to the motor;
determine a third force applied to the object by the user based on the first force and the second force; and
adjust the current applied to the motor at least partially based on the third force applied to the object.

2. The device in claim 1, wherein second finger is the thumb of the user, and the first finger is a finger different from the thumb.

3. The device in claim 1, further comprising:
an actuator interface that couples the motor to the wearable component; and
a guide band that is configured to be worn around a hand of the user and retains the actuator interface adjacent to the hand such that when the second force is applied by the motor, the guide band causes the actuator interface to bend the first finger toward the hand.

4. The device in claim 3, wherein the actuator interface is a metal band or a string.

5. The device in claim 1, wherein the wearable component comprises a strap around a user's finger.

6. The device in claim 1, wherein:
when driven in a first direction, the motor applies the second force to aid closing of the first finger; and
when driven in a second direction, the motor applied the second force to aid opening of the finger.

7. The device in claim 1, wherein the at least one force sensor comprises a strain gauge.

8. The device in claim 1, wherein the control system applies a curve fitting algorithm to determine the second force.

9. The device in claim 8, wherein the curve fitting algorithm is an exponential least squares curve fitting algorithm.

10. The device in claim 1, further comprising a second force sensor located on the palm side of a third finger of the user, the second force sensor operative to measure a fifth force applied to an object by the user.

11. The device in claim 1 further comprising an input/output interface.

12. The device in claim 1, further comprising an external pack housing the motor and the control system.

13. The device in claim 12, wherein the external pack comprises at least one attachment mechanism to secure the external pack to the user or user's medical equipment.

14. The device in claim 12, further comprising:
an actuator interface that couples the motor to the wearable component; and
a guide band that is configured to be worn around a hand of the user and retains the actuator interface adjacent to the hand such that when the second force is applied by the motor, the guide band causes the actuator interface to bend the first finger toward the hand.

15. The device in claim 12, wherein a portion of the actuator interface is encased in a tube connected to the external pack on a first end and the wearable component on a second end.

16. The device in claim 12, wherein the wearable component is a glove.

17. A method to determine different applied forces during the use of a device assisting in joint flexion, comprising:
receiving a first signal from an at least one force sensor indicating a first force applied to an object by a user;
receiving a second signal indicating a current applied to a motor;
determine a second force applied by the device to the object based on the current applied to the motor;
determine a third force applied to the object by a user based on the first force and the second force.

18. The method to adjust applied forces during the use of a device assisting in joint flexion, comprising:
receiving a first signal from a force sensor indicating a first force applied to an object;
receiving a second signal indicating a current applied to a motor;
determining a second force applied by the device to the object based on the current applied to the motor;
determining a third force applied to the object by the user based on the first force and the second force; and
adjusting the current applied to the motor at least partially based on the third force applied by the user.

19. The method in claim 18, further comprising adjusting the current applied to the motor at least partially based on a previous third force measurement applied by the user.

20. The method in claim 18, further comprising keeping the current applied to the motor constant until the user initiates a gesture.
